(19) United States
(12) Patent Application Publication
(10) Pub. No.: US 2003/0044868 A1
(43) Pub. Date: Mar. 6, 2003

(54) Method for detecting prion proteins in tissue samples

(76) Inventors: Abubakr Aslamkhan, Durham, NC (US); Donald Higgins, Franklinton, NC (US)

Correspondence Address: PARADIGM GENETICS, INC, 108 ALEXANDER DRIVE, P O BOX 14528, RTP, NC, 27709-4528, US

(21) Appl. No.: 09/924,812

(22) Filed: Aug. 8, 2001

(51) Int. Cl. G01N 33/53; G01N 33/542; G01N 33/537; G01N 33/543

(52) U.S. Cl. 435/7.92; 435/7.9; 436/515; 436/516

(57) ABSTRACT

Surprisingly, the present inventors have discovered that thermal denaturation of prion protein facilitates its detection by immunological methods. Accordingly, the present invention provides methods for the preparation and thermal denaturation of samples for prion detection, comprising: homogenizing a candidate sample and heating said sample in a buffer, preferably one with properties that aid stabilization of the denatured form of the protein. The methods described in this disclosure can be used in the detection of PrP^{Sc}. Such detection is useful for the diagnosis of transmissible spongiform encephalopathies. This method can be used with immunoassays of various formats, including, but not limited to, dot blot and western blot assays, which utilize polyclonal antibodies, monoclonal antibodies, antibody fragments, receptors, natural and synthetic ligands and other entities.

### FIELD OF THE INVENTION

The invention relates generally to prionics and molecular biology. In particular, the invention relates to methods for the immunodetection of prion proteins, specifically in tissue samples.

### BACKGROUND OF THE INVENTION

Transmissible spongiform encephalopathies (TSE) (which include Creutzfeldt-Jakob Disease (CJD) in humans, Scrapie in sheep, Bovine Spongiform Encephalopathy (BSE) in cattle, and Chronic Wasting Disease (cwd) in deer and elk) are fatal neurodegenerative disorders caused by an aggregation of a protein known as a prion on the surface of neurons. Prion (PrP) is a small membrane bound glycoprotein that is anchored in the plasma membrane at the C-terminus by a glycosylphosphatidylinositol moiety (Stahl et al. (1987) Cell 51: 229-40 (PMID: 2444340)). This protein has been shown to be present on the surface of neurons as well as on certain other cell types. The amino acid sequence of prion is highly conserved among various species (Schatzl et al. (1995) J Mol Biol 245: 362-74 (PMID: 7837269)). Although an exact function remains elusive, prion proteins may play a role in signal transduction (Moulliet-Richard et al. (2000) Science 289: 1925-8 (PMID: 10988071)). Structural studies using techniques such as X-ray crystallography and solution NMR have shown that prion can exist in two conformational states (Li et al. (2000) J Mol Biol 301: 567-73 (PMID: 10966770); Riek et al. (1996) Nature 382: 180-2 (PMID: 8700211); Zahn et al (2000) Proc Natl Acad Sci USA 97: 145-50 (PMID: 10618385)). In its normal form, designated by PrP, the molecule may be divided into two regions. The first 90 amino acids of the N-terminal region comprise a highly flexible part of the molecule (Borman (1998) Chemical & Engineering News 2: 1-19). The C-terminal region consists of 3 α-helices and two short anti-parallel β-strands. This part of the molecule also contains two N-glycosylation sites. The other conformational state of prion is characterized by the presence of four β-strands of equal length and two α-helices in the C-terminal half. This form, which is often referred to as PrP^{Sc}, is highly insoluble and partially resistant to digestion with proteinase K (Zahn et al. (2000) Proc Natl Acad Sci USA 97: 145-50 (PMID: 10618385)). This conformational state is associated with transmissible spongiform encephalopathies. Experimental evidence suggests that PrP^{Sc} forms a heterodimer with endogenous PrP and causes the latter to refold into β-sheet conformation. The newly formed PrP^{Sc} molecules then aggregate resulting in the formation of plaques on the cell membrane (Wisniewski et al (1998) Amyloid 5: 212-24 (PMID: 9818059)). The exact mechanism by which PrP^{Sc} invades the tissues of the central nervous system is not known. However, PrP^{Sc} accumulates in lymphatic organs of animals that have been inoculated with it at peripheral sites, including the stomach. B-lymphocytes may play a crucial role in the transport of PrP^{Sc} across the blood brain barrier (Horiuchi et al. (1995) J Gen Virol 76: 2583-7 (PMID: 7595362); Brown (1997) Nature 390: 662-3 (PMID: 9414158)).

Since nvCJD (new variant Creutzfeldt-Jakob disease) can be transmitted to humans by eating contaminated meat products, and also possibly through blood transfusion (Houston et al. (2000) Lancet 356: 999-1000 (PMID: 11041403)), screening livestock and blood donors for the presence of PrP^{Sc} is extremely important. This necessitates the development of diagnostic procedures that are fast, reliable, and sensitive. A wide variety of techniques have been employed for the detection of PrP^{Sc} in animal tissue. These range from dot blot (Serban et al. (1990) Neurology 40: 110-17 (PMID: 1967489)) and Western blot (Lee et al (2000) J Virol Methods 84: 77-89 (PMID: 10644089); Schaller et al. (1999) Acta Neuropathol (Berl) 98: 437-43 (PMID: 10541864)) assays to various ELISA formats (Grathwohl et al. (1997) J Virol Methods 64: 205-16 (PMID: 9079766); Safar et al. (1998) Nat Med 4: 1157-65 (PMID: 9771749); Meyer et al. (1999) J Virol 73: 9386-92 (PMID: 10516047)). To date, only one monoclonal antibody that reacts selectively with the native conformation of PrP^{Sc} has been described (Korth et al. (1997) Nature 390: 74-7 (PMID: 9363892)). This antibody has not yet been used as a diagnostic reagent. Virtually all of the antibodies against prion recognize linear epitopes (Betemps and Baron (2001) Biochem Biophys Res Commun 281: 101-8 (PMID: 11178966)). As a result, protein molecules have been denatured prior to interaction with the antibody. This task is accomplished by employing denaturing reagents like guanidine salts in conjunction with heat. Meyer, et al ((1999) J Virol 73: 9386-92 (PMID: 10516047)) denatured PrP^{Sc} present in BSE positive brain tissue by heating at 150° C. in buffer containing 0.1M sucrose. However, the authors found that thermal treatment alone did not result in sufficient unfolding of the prion protein for binding to antibodies, and that incorporation of 0.13M guanidine thiocyanate in the buffer was necessary for complete exposure of internal epitopes.

This is in stark contrast to the inventors' findings and method, which minimizes and can even eliminate the use of chemical denaturing agents for interaction with prion linear epitopes. Since chemical denaturing agents may also be detrimental to the antibodies, samples treated with such denaturing agents may need to be diluted prior to immunoassay. Such dilution may limit the amount of sample that can be used in the assay and hence affect its sensitivity. By minimizing or even eliminating chemical denaturing reagents the overall assay is also simplified.

To date there do not appear to be any publications describing thermal denaturation methods at ambient pressure without the use of acids or guanidine salts as an aid to prion detection. Thus, the prior art has not suggested this approach to preparing samples for prion detection. It would be desirable to use a method which provides speed, reliability, and an improved range of sensitivity for diagnosing the presence of a serious health threat.

### SUMMARY OF THE INVENTION

Surprisingly, the present inventors have discovered that a method for detecting, in the absence of guanidine salts and acids, the presence of prion protein in a tissue sample at ambient pressure comprising: heating said tissue sample to a temperature effective to denature said prion protein, and detecting said denatured prion protein. Such thermal denaturation of prion protein facilitates its detection by immunoassays of various formats, including, but not limited to, dot blot and western blot assays, which utilize polyclonal antibodies, monoclonal antibodies, antibody fragments, receptors, natural and synthetic ligands and other entities. Accordingly, the present invention provides methods for the preparation and thermal denaturation of tissue samples for prion detection. The methods described in this disclosure can be used in the detection of PrP^{Sc}. Such detection is useful for the diagnosis of transmissible spongiform encephalopathies.

### BRIEF DESCRIPTION OF THE DRAWINGS

FIG. 1 shows the thermal denaturation profile for prion.

FIG. 2 shows the denaturation of prion protein as a function of time.

FIG. 3 is an analysis of a panel of sheep brain homogenates.

FIG. 4 is an analysis of deer tonsil homogenates.

### DETAILED DESCRIPTION OF THE INVENTION

Definitions

The term “acid” refers to a fundamental category of compounds whose water-based solutions have a sour taste, turn blue litmus paper red, and can combine with metals to form salts. Specific types of acids include: Arrhenius acids: any chemical that increases the number of free hydrogen ions (H+) when they're added to a water-based solution—the more free hydrogens produced, the stronger the acid; Brönsted or Brönsted-Lowry acids: any chemical that acts as a proton donor in a chemical reaction; and Lewis acids: any chemical that accepts two electrons to form a covalent bond during a chemical reaction. As used herein, Arrhenius acids have a pH lower than 3.0.

The term “ambient pressure” refers to current atmospheric pressure in the surrounding air.

The term “antibody” refers to an immunoglobulin capable of specific combination with the antigen that caused its production, and as used herein encompasses an antibody, and fragments and conjugates thereof. An antibody may be monoclonal or polyclonal, and present in a variety of media including, but not limited to, serum or supernatant, or in purified form.

The term “antigen” refers to a substance that elicits an immune response particularly the formation of specific antibodies capable of binding to it.

The term “binding” refers to a noncovalent interaction that holds two molecules together. For example, two such molecules could be an enzyme and an inhibitor of that enzyme. Noncovalent interactions include hydrogen bonding, ionic interactions among charged groups, van der Waals interactions and hydrophobic interactions among nonpolar groups. One or more of these interactions can mediate the binding of two molecules to each other.

The term “BSE” refers to bovine spongiform encephalopathy, a form of TSE.

The terms “CJD” or “CJS” refer to Creutzfeldt-Jakob Syndrome also known as Creutzfeldt-Jakob Disease, the most common form of TSE which affects humans; usually causes dementia in people around 65 years of age.

The term “calorimetric assay” refers to an assay based on estimating the color of an experiment's result, e.g. by comparing it with the colors of standard solutions. Usually the reaction is designed to produce a colour so that the colour intensity can be used to estimate the amount of antibody bound to the antigen and hence the amount of antigen present. Such an assay may be qualitative or quantitative, though generally the latter.

The term “cwd” refers to chronic wasting disease, a form of TSE.

The term “denatured prion protein” refers to prion protein that has undergone denaturation or has an epitope permitting immunodetection. Denaturation usually involves unfolding of the polypeptide chains of proteins and of the double helix of nucleic acids, with loss of secondary and tertiary structure; it is caused by heat (thermal denaturation), chemicals, and extremes of pH. The loss of secondary and tertiary structure can aid antibodies raised with linear (as opposed to conformational) epitopes as antigens in binding.

The term “detergent” refers to detergents and surfactants. Detergents are compounds that can permit nonpolar substances to go into solution in polar solvents, such as water. Typically they are composed of a hydrocarbon oil-soluble part and a water-soluble part. Alkyl sulphonates are common examples. There are several classes of detergent, such as anionic detergents, for which the active part is a negative ion; cationic detergents, which usually have a long hydrocarbon chain connected to a positive ion; and nonionic detergents, which have nonionic polar groups of the type —C2H4—O—C2H4—OH, which form hydrogen bonds with water. Surfactants are substances that lower the surface tension of a liquid: allowing easier penetration and spreading, they are often known as wetting agents. Surfactants active in water are usually organic substances, e.g. alcohols, and soaps, the molecules of which contain both a water-soluble (hydrophilic) and a water-repelling (hydrophobic) portion; the latter forces the molecules to the surface.

The term “DMSO” refers to dimethyl sulfoxide.

The term “dot blot” herein refers to a method for detecting a protein. The sample is adsorbed onto a solid support, such as a nitrocellulose filter, and an appropriate antibody probe, which binds to the protein being investigated, is added. After a period of incubation any excess probe is washed off and the protein under investigation can be detected by various methods known to those in the art for detecting antibodies.

The term “EDTA” refers to ethylenediaminetetraacetic acid.

The term “electrochemiluminescence” or “electrogenerated chemiluminescence” (ECL) refers to a form of chemiluminescence (CL) in which the light emitting chemiluminescent reaction is preceded by an electrochemical reaction. The advantages of CL are retained, but the electrochemical reaction allows the time and position of the light emitting reaction to be controlled. By controlling the time light emission can be delayed until events such as immune or enzyme catalyzed reactions have taken place. Although similar control can be exercised over alternative detection methods such as fluorescence the equipment is considerably more sophisticated and expensive. Control over position can be used to confine light emission to a region which is precisely located with respect to the detector, improving sensitivity by increasing the ratio of signal to noise. A good example of this is the combination of ECL with magnetic bead technology, which allows bound label to be distinguished from unbound label without a separation step. Control over position could also be used to determine the results of more than one analytical reaction in the same sample by interrogating each electrode in an array, either in sequence, or simultaneously using a position sensitive detector.

The term “ELISA” means enzyme-linked immunosorbent assay.

The term “enzyme-linked immunoassay” or “EIA” refers to an immunoassay coupled to an enzyme or substrate for use immunodetection. An ELISA is a form of EIA, though EIA also encompasses non-solid phase immunoassays and other formats of solid phase assays as well.

The term “epitope” refers to a molecular region on the surface of an antigen capable of eliciting an immune response and of binding with the specific antibody produced by such a response.

“Fatal Familial Insomnia” refers to another form of TSE that affects humans; dementia follows sleeping difficulties.

“GSS” refers to Gerstmann-Straussler-Scheinker disease, a form of TSE that affects humans.

The term “HCl” refers to hydrochloric acid.

The term “HEPES” refers to 4-(2-hydroxyethyl)-1-piperazineethanesulfonic acid.

The term “immune response” refers to the series of molecular events that are elicited when an antigen is encountered by the immune system. These include the expansion of B- and T-cells and the production of antibodies.

The term “immunoassay” refers to one of a number of techniques for the determination of the presence or quantity of a substance, esp. a protein, through its properties as an antigen or antibody. The binding of antibodies to antigen is often followed by tracers, such as fluorescence or (radioactive) radioisotopes, to enable measurement of the substance. Immunoassays have a wide range of applications in clinical and diagnostic testing. An example is solid-phase immunoassay in which a specific antibody is attached to a solid supporting medium, such as a PVC sheet. The sample is added and any test antigens will bind to the antibody. A second antibody, specific for a different site on the antigen, is added. This carries a radioactive or fluorescent label, enabling its concentration, and thus that of the test antigen, to be determined by comparison with known standards.

The term “immunodetection” refers to detecting the presence of an antigen by an antibody.

The term “immunoelectrophoresis” refers to electrophoretic separation of proteins followed by identification by the formation of precipitates through specific immunologic reactions.

The term “immunofluorescence” refers to the labeling of antibodies or antigens with fluorescent dyes, especially for the purpose of demonstrating the presence of a particular antigen or antibody in a tissue sample.

The term “immunohistochemisty” refers to histochemical localization of immunoreactive substances using labeled antibodies as reagents.

The term “immunoreactivity” encompasses both immunogenicity (the ability to elicit an immune response) and the degree of a particular antigen's binding interaction with an antibody or antibodies.

“Kuru” refers to another form of TSE that affects humans; this disease has only been observed in the Kuru people of New Guinea and has not been seen since the Kuru stopped the cannabilistic practices that caused an outbreak during the mid 1980's.

“MCD” refers to Mad Cow Disease, the generic name for Bovine Spongiform Encephalopathy (BSE), a TSE.

The term “nvCJD” refers to new variant Creutzfeldt-Jakob disease, a TSE.

The term “PBS” refers to phosphate buffered saline.

The “percent (%) sequence identity” between two polynucleotide or two polypeptide sequences can be determined according to the either the BLAST program (Basic Local Alignment Search Tool; Altschul and Gish (1996) Meth Enzymol 266:460-480 and Altschul (1990) J Mol Biol 215:403-410) in the Wisconsin Genetics Software Package (Devererreux et al. (1984) Nucl Acid Res 12:387), Genetics Computer Group (GCG), Madison, Wis. (NCBI, Version 2.0.11, default settings) or using Smith Waterman Alignment (Smith and Waterman (1981) Adv Appl Math 2:482) as incorporated into GeneMatcher Plus™ (Paracel, Inc., http://www.paracel.com/html/genematcher.html; using the default settings and the version current at the time of filing). It is understood that for the purposes of determining sequence identity when comparing a DNA sequence to an RNA sequence, a thymine nucleotide is equivalent to a uracil nucleotide.

By “polypeptide” is meant a chain of at least four amino acids joined by peptide bonds. The chain may be linear, branched, circular or combinations thereof. The polypeptides may contain amino acid analogs and other modifications, including, but not limited to glycosylated or phosphorylated residues.

The term “prion protein” refers to the infective agent(s) believed to be responsible for Kreutzfeld-Jacob disease, kuru, GSS, Fatal Familial Insomnia, and possibly other degenerative diseases of the brain in human beings, scrapie in sheep, and bovine spongiform encephalopathy (BSE). The term can also be used to encompass or refer to the normal cellular form of the protein. Transmission occurs by ingestion of infected tissue. Prion stands for “proteinaceous infectious particle.” Unique as a pathogen, prions are neither viral nor bacterial and contain no nucleic acids; instead, they are simply a protein. Fifteen years ago dogma held that any infectious particle (as prions are) must contain nucleic acids in either the form of DNA or RNA in order to reproduce once they had successfully invaded a host. Scientists first became aware that spongiform encephalopathies were caused by an atypical pathogen when extracts that were taken from the brains of sheep were exposed to ultraviolet and ionizing radiation (conditions which usually destroy nucleic acids) retained their ability to transmit the disease when injected into a healthy animal.

Most diseases are caused by either a genetic mutation or by a pathogen; however, spongiform encephalopathies can occur one of three ways: (1) the spontaneous conversion of normal prions into abnormal prions; (2) the mutation of the gene that directs the formation of prions; (3) transmission through consumption of infected material.

The main difference between the protein that is produced by the normal gene and the prion that is produced by the abnormal gene is conformational (there is also a difference of 1 amino acid). That is, the shapes of the two molecules are different. The normal protein consists mainly of alpha helices (regions in which the protein backbone twists into a specific kind of spiral) while the mutant prion is shaped in beta strands (regions in which the backbone is fully extended) that clump together to form beta sheets.

The prion protein seeks out the normal protein and is able to somehow convert the normal protein into a prion. Once this process begins, it goes to completion; all of the normal protein is converted into the prion protein. After this has occurred, death is imminent.

One of the biggest threats that diseases caused by prions pose is their potential to cross the species barrier and simultaneously infect several species while retaining the same chemical and structural form. MCD was probably passed from sheep to cows through contaminated feed (sheep meal) that the cows were given to supplement their diet. The more the sequence of the infectious prion of one species resembles the prion protein sequence of the host, the more likely it is that the host will acquire the prion disease. The infectious prion protein of sheep and the protein created by cows from their prion gene differs in only 7 positions in the amino acid sequence.

The term “probe” refers to a tool for detection.

“Proteinase K” (PROK) is a serine protease with broad specificity towards aliphatic, aromatic and other hydrophobic amino acids. PROK has a molecular weight of 27,000 daltons and is Ca2+ dependent. It is not inactivated by metal ion chelating agents such as EDTA, sulfhydryl reagents, p-chloromercuribenzoate (PCMB), tosyl-L-lysine chloromethylketone (TLCK), or 1-Chloro-3-tosylamido-4-phenyl-2-butanone (TPCK). It also retains activity in SDS (<5 mg/ml). It can be inhibited by phenylmethylsulfonyl fluoride (PMSF) or diisopropylfluorophosphate (DFP).

The term “PrP” refers to prion, a small membrane bound glycoprotein anchored in the plasma membrane at the C-terminus by a glycosylphosphatidylinositol moiety. Prion has been shown to be present on the surface of neurons as well as on certain other cell types. The amino acid sequence of prion is highly conserved among various species.

The term “PrP^{Sc}” refers to a highly insoluble form of prion that is partially resistant to digestion with proteinase K and associated with transmissible spongiform encephalopathies.

The term “PVC” refers to Poly(vinyl chloride).

The term “radioimmunoassay” refers to an immunoassay using a radionucleotide to label the antigen or antibody. It is a very sensitive technique. Isotopes such as ³H, ¹³¹I or ¹²⁵I can be used. ¹²⁵I is often the isotope of choice because it emits gamma rays and thus produces a strong signal, leading to highly sensitive analyses.

The term “rPrP” refers to bovine recombinant prion protein.

The term “Sarkosyl” refers to the detergent also known as “N-lauroylsarcosine”.

The term “scrapie” refers to Scrapie is a TSE affecting sheep and goats. It is thought to be widespread in Europe, the US and the Middle East, but it is today notably absent in Australia and New Zealand. The earliest records on scrapie date back to the early 18^{th} century. The name scrapie comes from the tendency of the diseased sheep to scrape off their wool. Other clinical symptoms include difficulties in walking and loss of coordination, which is reflected in the names ‘Traberkrankheit’ (trotting disease) and ‘la tremblante’ (the tremble), names that were originally used in Germany and in France, respectively.

The term “SDS” refers to sodium docecyl sulfate.

The term “solid-phase immunoassay” refers to an immunoassay performed in a heterogeneous manner in which either the antigen or antibody is bound to a solid surface such as a microtitre plate or test tube, as opposed to a homogeneous manner in which all reactions are performed in the liquid phase. Examples are ELISA and Western blotting.

The term “surface plasmon resonance-based technology” refers to surface plasmon resonance (SPR) immunoassays, which are based on the biological input of antibody-antigen binding, while the measurable output is either a resonance angle or refractive index value. Surface plasmon resonance (SPR) is an optical phenomenon that is used to measure changes in the solution concentration of molecules at a surface. This signal arises in thin metal films under conditions of total internal reflection. This signal depends on the refractive index of solutions in contact with the surface. Molecules in solution exhibit changes in refractive index and thus give rise to a measurable SPR signal if an interaction occurs. Typically, protein or DNA is immobilized by one of several possible methods onto a carboxymethylated dextran-gold surface. The interacting protein of interest is injected over the surface and the kinetics of binding are measured in real time. SPR is a relatively new method for characterizing protein-protein interactions. BIAcore was the first to make this technology commercially available circa 1990. It allows the determination of which molecules interact, how strong the interaction is, as well as how fast, and of the concentrations.

The term “tissue sample” refers to a quantity of body fluid, cells, tissue, or organ, and combinations thereof.

The term “Triton X-100” refers to the detergent also known as “octylphenol ethylene oxide condensate” or “Octoxynol-9”.

The term “TSE” refers to “Transmissible Spongiform Encephalopathies”; also called “spongiform encephalopathies”, these diseases are characterized by physical degradation of the brain, resulting in dementia and loss of coordination. Although these diseases are relatively rare, the fatality rate of any of the above mentioned diseases is 100%; no cures exist for any of them at present. While the symptoms of these diseases vary slightly from specie to specie, their distinguishing characteristic is the physical degradation of the brain. Most evident during a postmortem examination, the brain of an individual who had an encephalopathy is riddled with holes; it is porous (like a sponge) particularly in the cerebellum (which controls coordination and involuntary muscle contraction) and the cerebrum (which controls higher order thought processes). The cause of TSE's in mammals has been found to be changes in a protein called PrP which is found in nerve cells throughout the brain. This mutated PrP (PrP^{Sc}) is commonly called a prion (PREE-on), which stands for “proteinaceous infectious particle.”

The term “Tween 20” refers to the detergent also known as “sorbitan mono-9octadecenoate poly(oxy-1,1-ethanedlyl)” or “polyoxyethylene sorbitan monolaurate”.

The term “USDA” refers to the United States Department of Agriculture.

The term “Western blot” refers to an immunoassay for determining very small amounts of a particular protein in tissue samples or cells. The sample is subjected to electrophoresis on SDS-polyacrylamide gel to separate constituent proteins. The resultant protein bands are then ‘blotted’ onto a polymer sheet. An antibody specific for the target protein is added; this binds to the protein, and can then be detected by a variety of methods known to those in the art. The name is derived by analogy to that of Southern blotting.

The term “2E11” refers to a specific monoclonal antibody used in the detection of prion.

The term “6H4” refers to a specific monoclonal antibody used in the detection of prion.

### EMBODIMENTS OF THE INVENTION

The inventors have discovered a rapid method for the denaturation of prion protein (PrP and PrP^{Sc}) using thermal energy. Heat-unfolded prion protein can then bind to anti-prion antibodies that recognize linear epitopes. Prion is extracted from a tissue sample and treated with proteinase K by following protocols outlined in the literature and herein. The protein homogenate or solution is then heated for between about 2 and 20 minutes, preferably between about 5 and 15 minutes, most preferably for about 10 minutes, in a disposable tube on a heating block set at a temperature between about 75° C. and about 100° C., more preferably between about 80° C. and about 90° C., most preferably about 85° C., and allowed to cool down to room temperature, after which it can be used in immunoassays of various formats.

The method of the present invention can be carried out in the absence of acids and guanidine salts. However, the tissue sample may still allow for small or trace quantities of acids or guanidine salts whose amounts would be insufficient to per se denature the prion protein.

This method of denaturation correlates with the method in which guanidine HCl is employed as a chemical denaturing agent for unfolding the prion protein. Heat denaturation alone in sharp contrast to chemical denaturation enabled the detection of scrapie prion in the brain homogenate from a sheep that had not exhibited clinical symptoms of the disease but was found to be infected (pre-clinical stage) with scrapie by immunohistochemistry studies on the brain tissue (see Example 5 below). The heat denaturation procedure was successfully used to detect the disease form of prion protein in brain and tonsil tissue samples from scrapie positive sheep (Example 5 below), and chronic wasting disease positive deer (Example 6 below), respectively. The exposure scrapie and cwd PrP^{Sc} epitopes for interaction with monoclonal antibodies 6H4 and 2E11 through thermal denaturation in principle can be extended to other antibodies. This method can also be extrapolated for use in the denaturation of prion from other species for detection, for example BSE in cattle, nvCJD in humans, etc.

The prion protein may have the amino acid sequence of a naturally occurring prion protein found in an animal, plant, or microorganism, or may have an amino acid sequence derived from a naturally occurring sequence. Polypeptides having at least 80% sequence identity with a prion protein are also within the scope of the methods of the invention. Preferably, the sequence identity is at least 85%, more preferably the identity is at least 90%, most preferably the sequence identity is at least 95% or 99%. Preferably the prion protein is a mammalian prion protein.

In various embodiments, the prion protein can be from mammals such as humans, deer, elk, sheep, cows, alpacas, buffaloes, bears, aardvarks, horses, donkeys, camels, antelopes, rhinoceroses, caribou, dogs, alpacas, armadillos, cats, coyotes, gazelles, hedgehogs, elephants, ferrets, goats, llamas, lions, leopards, whales, oxen, horses, moose, rabbits, gorillas, pigs, tigers, raccoons, chimpanzees, water buffalo, weasels, rodents, skunks, wolves, zebras, and the like.

In various embodiments, the tissue sample can be cells, body fluids, or tissues, and combinations thereof such as blood, milk, muscle tissue, urine, chondrocytes, beef, pork, cheek cells, brain tissue, liver, tonsils, saliva, adipose tissue, hearts, pig' feet, lungs, toenails, hair, skin, hamburger, teeth, and the like.

Any technique for detecting the presence of a specific protein may be used in the methods of the invention. Methods for detecting protein expression include, but are not limited to, immunodetection methods such as Western blots, His Tag and ELISA assays, polyacrylamide gel electrophoresis, mass spectroscopy and enzymatic assays. For example, the ligand and target are combined in a buffer. Many methods for detecting the binding of a ligand to its target are known in the art, and include, but are not limited to the detection of an immobilized ligand-target complex or the detection of a change in the properties of a target when it is bound to a ligand.

### EXAMPLE 1

### PrP Deer Tonsil Extraction Protocol

### Materials/Equipment

1. Homogenization Buffer: 50 mM Tris, pH 7.5, 100 mM NaCl, 4% (v/v) Triton X-100, 0.5% (w/v) Sarkosyl

2. Collagenase

3. DNAse I

4. Deer or elk tonsils

5. Microfuge tubes, 1.5 ml

6. Conical tubes, 50 ml

7. Polytron or equivalent (homogenizer made by Brinkmann, Westbury, N.Y.)

8. Omni-Tip Disposable Rotor-Stator Generator Probes, 110 mm (for use with homogenizer, Omni International, Inc., Warrenton, Va.)

9. Dry Block Heater

### Procedure for Extracting PrP from Deer Tonsil

1. Weigh 0.1-0.5 g of deer or elk tonsil and transfer to a 1.5 mL microfuge tube.

2. Add 5 volumes of Homogenization Buffer per weight of tonsil.

3. Add collagenase to a final concentration of 3.0 mg/ml

4. Add DNAse I to a final concentration of 0.25 mg/ml.

5. Mix tube by vortexing and incubate at 37° C. for 5 hrs.

6. Transfer the suspension to a 50 ml conical tube and add 0.5 ml of Homogenization Buffer.

7. Homogenize the sample for 5 minutes using a Polytron equipped with a 110 mm disposable Omni Tip.

### EXAMPLE 2

### ELISA Protocol for Scrapie Detection

### Materials/Equipment

1. Anti-PrP Monoclonal Antibody-coated Stripwells (store at 2-8° C.): black, plastic 96 well microplate coated with mouse monoclonal antibody 6H4 (Prionics AG, Switzerland). 6H4 is coated in phosphate buffered saline (PBS), pH 7.4 at concentrations ranging from 5-10 ug/ml for 16-20 hrs at 4° C. The coating solution is then removed and the wells are blocked with PBS containing 0.2% (w/v) of I-Block (Applied Biosystems, Foster City, Calif.) for 1-1.5 hrs at 37° C. in the presence of a dessicant.

2. Homogenization Buffer (store at 2-8° C.): a solution containing 3 mM dithiothreitol, 2% N-lauryl sarcosine, 250 mM sucrose, 15 mM EDTA buffered with 20 mM HEPES, pH 7.0.

3. Homogenization Buffer Concentrate: 5× concentrate; mix 200 ml of concentrate with 800 ml of deionized water. Store at 2-8° C.

4. Digestion Buffer (store at 2-8° C.): a solution containing 3 M guanidine chlorhydrate and 1 g/L N-methylisothiazolone.

5. Proteinase K (store at 2-8° C.): 2 mg/ml in a 50% glycerol solution buffered with 10 mM Tris, pH 7.5.

6. Proteinase K Stop Solution (store at 2-8° C.): 40 mM Pefablock Protease Inhibitor (Boehringer-Mannheim Corp., Indianapolis, Ind.).

7. Detection Antibody (store at 2-8° C.): mouse monoclonal antibody 2E11 (Prionics AG, Switzerland) conjugated to horseradish peroxidase or another enzyme or fluorescent label.

8. Dilution Buffer (store at 2-8° C.): phosphate buffered saline, pH 7.4 containing 0.02% (w/v) I-Block (Applied Biosystems, Foster City, Calif.), 0.1% (v/v) DMSO.

9. Bovine Recombinant PrP (Prionics AG, Switzerland): rehydrate with 1.0 ml of sterile deionized water. Mix well. Divide into 75 ul aliquots and freeze at −80° C. Thaw 1 aliquot and store at 2-8° C. Discard aliquot after 1 week.

10. Chemiluminescent Substrate: store at 2-8° C.

11. Phosphate Buffered Saline: store packets at 10-30° C. Solution can be used for up to 4 weeks if stored at 2-8° C.

12. Plate Sealers.

13. Microdilution tubes and caps: for storage and heating of samples. Samples can be directly loaded onto Capture Plate from these tubes.

14. Polytron or equivalent: Omni GLH Homogenizer (Omni International, Inc., Warrenton, Va.)

15. Omni-Tip Disposable Generator Probes, 110 mm (Omni International, Inc., Warrenton, Va.)

16. Plate Washer

17. Luminescent Plate Reader

18. Dry Block Heater, capable of 85° C. with blocks for 1.5 ml and 0.5 ml tubes

19. Various pipettors and pipets

20. Pipet tips

21. Tween 20

22. Conical tubes: 50 ml and 15 ml

23. Microfuge tubes: 0.5 ml and 1.5 ml

24. Bleach, for decontamination

Procedure for Detecting Scrapie Using the ELISA Assay

Note: allow all reagents to warm to room temperature before use.

1. Prepare 10% brain tissue homogenates by adding 10 volumes of 1× Homogenization Buffer per 0.1-0.5 g of each tissue. Homogenize each tissue in a Polytron for 3 minutes.

2. Digest each sample homogenate with proteinase K: add 10 ul of Digestion Buffer and 10 ul of Proteinase K per 100 ul of 10% tissue homogenate. Mix each sample by vortexing. Incubate for 1 hour at 47° C. At the end of the incubation, add 10 ul of Proteinase K Stop Solution and mix by vortexing.

3. Prepare rPrP Control Samples from the 100 ug/ml Stock rPrP Solution. prepare a 1:10 pre-dilution (10.0 ug/ml) of the 100 ug/ml Stock rPrP Solution by adding 20 ul to 180 ul of 1× Homogenization Buffer. Mix well. Use the 10.0 ug/ml rPrP Solution to prepare the High. Medium, and Low Control Samples in 1× Homogenization Buffer using Table 1 below as a guide. Mix each Control Sample well.

TABLE 1

| | rPrP | | Pre- | Volume | Volume | Total |
|---|---|---|---|---|---|---|
| Sample | Conc. | rPrP | dilution | rPrP (10 | Hom. Buffer | Volume |
| ID | ng/ml | pg/well | factor | ug/ml) ul | ul | ul |
| High | 40.0 | 1600 | 10 | 40.0 | 9960 | 10000 |
| Medium | 20.0 | 800 | 10 | 20.0 | 9980 | 10000 |
| Low | 10.0 | 200 | 10 | 5.0 | 9995 | 10000 |

4. Transfer a sufficient volume of each digested sample, assay blank, and rPrP control sample to the appropriate tube. Cap tubes tightly and heat at 85° C. for 10 min. Promptly remove each tube after 10 minutes and allow to cool to room temperature.

5. Dilute the Detection Antibody to a final working dilution of 1:1500 in Dilution Buffer. Prepare a volume of diluted Detection Antibody sufficient for the number of ELISA wells required. Mix well, but avoid foaming.

6. Wash the number of Capture Antibody stripwells required for duplicate testing. Wash each well four times with 300 ul of Phosphate Buffered Saline, pH 7.4 containing 0.05% Tween 20.

7. Transfer 60 ul of diluted Detection Antibody per well of the washed Capture Antibody Plate. Allow two wells for each sample, PrP Control Sample, and Assay Blank.

8. Transfer 40 ul of each sample, PrP Control Sample, and Assay Blank to the well of the Capture Antibody Plate. Repeat this step in an adjacent well so that all testing is done in duplicate. Mix samples just prior to transferring to the Capture Plate.

9. Seal the plate with a plate sealer and place on a shaker at 150 rpm for 1 hr at room temperature.

10. Near the end of the incubation time, prepare a sufficient volume of Chemiluminescence Substrate by mixing the 2 substrate components in a 1:1 ratio.

11. Wash the Capture Antibody Plate 5 times using 300 ul/well of Phosphate Buffered Saline, pH 7.4 containing 0.05% Tween 20.

12. Add 100 ul of Chemiluminescent Substrate per well of the Capture Antibody Plate.

13. Insert the plate into a luminometer and read.

### EXAMPLE 3

### Determination of Thermal Denaturation Profile for Prion

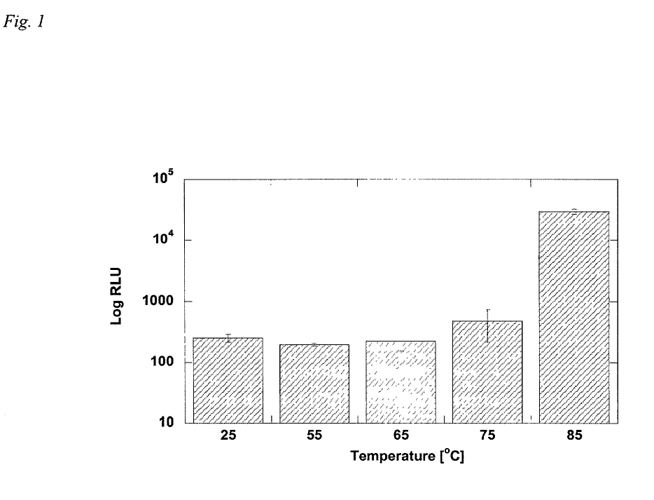

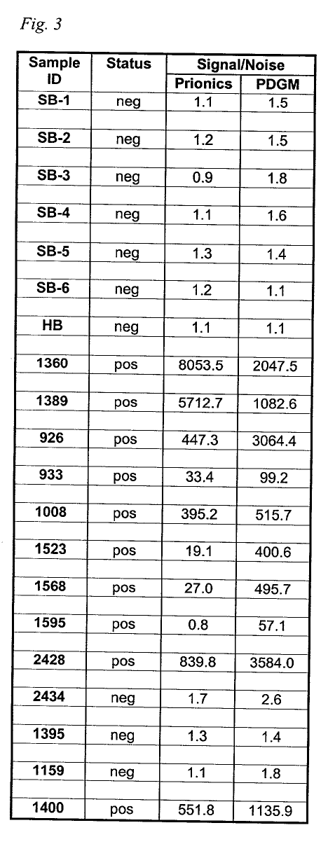

To find the temperature for optimal denaturation of PrP^{Sc}, scrapie positive sheep brain homogenate (Sample ID 1360, FIG. 3) was digested with proteinase K using the procedure described in Example 2. It was then diluted 14-fold in proteinase K digested negative brain pool (brain homogenate from six scrapie negative sheep were pooled on an equal volume basis). The tissue sample was heated to determine a temperature effective to denature said prion protein, and detect the denatured prion protein. Both the positive and negative brain pool samples were heated for about 10 minutes at various temperatures ranging from 25° C. to 85° C. Immunoreactivity of the denatured protein was then measured by ELISA. The results are shown in FIG. 1. The graph (FIG. 1) indicates that heating a tissue sample to a temperature of about 75° C. or greater can be used for thermal denaturation of the prion molecule.

### EXAMPLE 4

### Denaturation of Prion Protein as a Function of Time

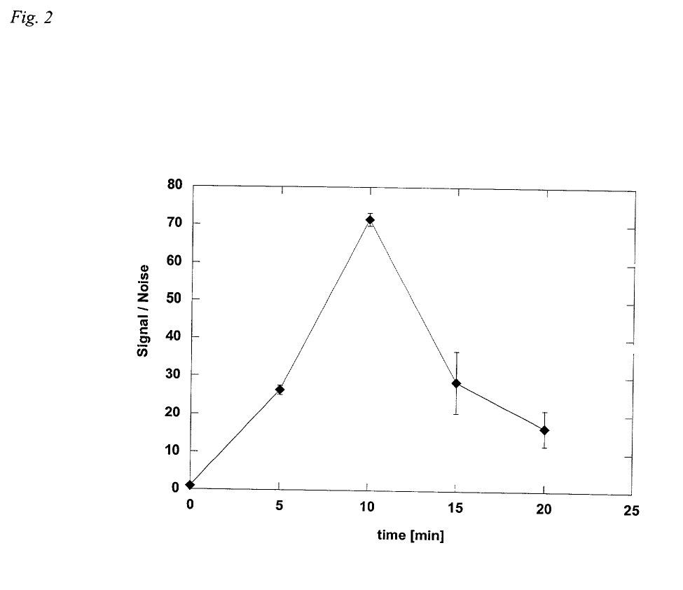

To find the optimal length of time to denature prion protein at 85° C., proteinase K digested scrapie positive sheep brain homogenate sample (Sample ID 1360) was diluted 10-fold in proteinase-K treated pooled normal brain homogenate. This sample was heated at 85° C. for 0 to 20 minutes. The results are shown in FIG. 2. Heating between 2 and 20 minutes results in a signal that is an order of magnitude above the background, with an optimum around 10 minutes. The decrease in immunoreactivity after 10 minutes is attributed to chemical degradation of the protein with continued exposure to elevated temperatures.

### EXAMPLE 5

### Analysis of a Panel of Sheep Brain Homogenates

Scrapie positive sheep brain samples were obtained from the USDA. Brain stem samples from normal sheep were purchased from a local slaughterhouse. Tissue samples were extracted and analyzed by following the protocol outlined in Example 2. Proteinase K digested tissue homogenate was heated to 85° C. in a buffer containing the detergent N-lauroylsarcosine alone or in combination with Triton X-100. The results are tabulated in FIG. 3. The data obtained by the ELISA agreed with the analysis of the samples using other techniques such as western blotting and immunohistochemisty (data not shown). Sample 1595 came from a sheep that was in the pre-clinical stage of infection. This sample was not detected as scrapie positive after standard chemical denaturation with guanidine (Prionics AG, Switzerland).

### EXAMPLE 6

### Analysis of Deer Tonsil Homogenates

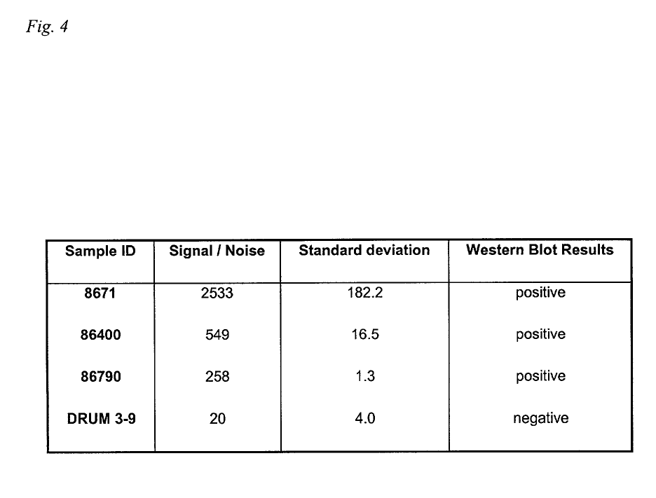

Deer tonsil samples from cwd infected and normal animals were provided by the USDA. Tissue samples were prepared and assayed for PrP^{Sc} by the procedure described in Example 1. The results are tabulated in FIG. 4. The ELISA results agreed both with western blotting analysis and immunohistochemistry studies conducted by the USDA (data not shown).

While the foregoing describes certain embodiments of the invention, it will be understood by those skilled in the art that variations and modifications may be made and still fall within the scope of the invention.

What is claimed is:

1. A method for detecting the presence of prion protein in a tissue sample comprising:
a) heating said tissue sample at ambient pressure, and in the absence of guanidine salts and acids, to a temperature effective to denature said prion protein, and
b) detecting said denatured prion protein.

2. The method of claim 1, wherein said temperature is between about 75° C. to about 100° C.

3. The method of claim 1, wherein said temperature is between about 80° C. to about 90° C.

4. The method of claim 1, wherein said temperature is about 85° C.

5. The method of claim 1, wherein prior to or during heating, said tissue sample is homogenized.

6. The method of claim 1, wherein prior to or during heating, said tissue sample is homogenized in a media containing at least one detergent.

7. The method of claim 6, wherein said detergent is octylphenol ethylene oxide condensate, polyoxyethylene sorbitan monolaurate, N-lauroylsarcosine, or combinations thereof.

8. The method of claim 1, wherein said sample is allowed to cool to room temperature prior to detecting the denatured protein.

9. The method of claim 1, wherein said prion protein is from a mammal.

10. The method of claim 1, wherein said prion protein is selected from the group consisting of cows, sheep, humans, deer, and elk.

11. The method of claim 1, wherein said detecting is performed using an immunoassay.

12. The method of claim 1, wherein said detecting is performed using an immunoassay is selected from the group consisting of ELISA, Western blot, dot blot, immunohistochemistry, radioimmunoassay, and immunofluorescence.

13. The method of claim 1, wherein said detecting is performed using an immunoassay selected from the group consisting of an enzyme-linked immunoassay, an immunoassay based upon detection using electrochemiluminescence, an immunoassay based upon surface plasmon resonance-based technology, a calorimetric immunoassay, a solid-phase immunoassay, and an immunoassay involving immunoelectrophoresis.
